(19) United States
(12) Patent Application Publication
(10) Pub. No.: US 2020/0116090 A1
(43) Pub. Date: Apr. 16, 2020

(54) VEHICLE OXYGEN-ENRICHED CABIN AIR SYSTEM

(71) Applicant: Reliable Energy Group Corp., Saugus, MA (US)

(72) Inventors: Jude Tam Van Tran, Tewksbury, MA (US); Gioankim Tran Dinh Quyen, Ho Chi Minh City (VN)

(21) Appl. No.: 16/599,495

(22) Filed: Oct. 11, 2019

Related U.S. Application Data
(60) Provisional application No. 62/745,048, filed on Oct. 12, 2018.

(51) Int. Cl. F02D 41/00; B60H 1/24; F02M 35/10; F02B 47/06; C25B 1/04; B01D 53/047; G01N 33/00

(52) U.S. Cl. CPC F02D 41/0002; B60H 1/24; F02M 35/10209; F02M 35/10301; F02B 47/06; F02M 35/10262; B01D 2259/4566; B01D 53/0476; G01N 33/0062; F02D 2200/04; B01D 2256/12; B01D 2257/102; C25B 1/04

(57) ABSTRACT

This invention provides a method and system for providing an oxygen-enriched air stream to the cabin and/or internal combustion engine of a vehicle. The benefits of such an approach include increased fuel efficiency and reduced emissions in the internal combustion engine and increased driver alertness and comfort for the vehicle operator. The oxygen-enriched air stream is provided by a membrane separation system, a pressure swing adsorption system, vacuum swing adsorption, or other methods. The delivered oxygen-enriched air is controlled to a prescribed level using sensors, valves, and controllers.

### CROSS-REFERENCE TO RELATED APPLICATION(S)

This application claims priority to, and the benefit of, co-pending U.S. Provisional Application 62/745,048, filed Oct. 12, 2018, for all subject matter common to both applications. The disclosure of said provisional application is hereby incorporated by reference in its entirety.

### FIELD OF THE INVENTION

The present invention relates to the operation of transportation vehicles. In particular, the present invention relates to systems and methods for providing oxygen-enrichment to the cabin air of a vehicle and providing oxygen to the combustion engine of the vehicle, both from the same source. By increasing the volume fraction of oxygen in the vehicle's cabin, the invention aids users by improving mental acuity, comfort, and alertness, and by increasing the oxygen to the combustion engine efficiencies and power are improved.

### BACKGROUND

Generally, breathing in air with an elevated amount of oxygen can result in improved alertness and other physiological effects. Increased driver alertness plays a significant role in increasing safety on the roads and therefore saving lives.

One conventional approach to elevating the concentration of oxygen in an inhaled stream is using compressed gas cylinder tank containing pure oxygen or elevated amounts of oxygen. However, supplying oxygen in this manner is not cost effective and necessitates continual replacement of the oxygen-providing system in the form of the compressed oxygen tank.

It has also been shown that operating an internal combustion engine using air enriched with additional oxygen has favorable characteristics in terms of increased fuel efficiency and reduced emissions. The increase in fuel efficiency by completely burning the unburned fuel in the combustion engine can be enough and sufficient to negate the cost to produce the oxygen-enrichment. In addition, the reduced emissions minimize the generation and release of toxic substances, such as Carbon Monoxide (CO) and Nitrogen Monoxide (NO) that could be inhaled by the drivers, passengers, and anyone near the vehicle.

### SUMMARY

While the above benefits are known, there remains a need for a system that efficiently generates elevated levels of oxygen for use in enriching air fed to both the cabin of the vehicle to benefit the driver, and/or intake air to the internal combustion engine to benefit fuel efficiency and reduce emission of toxic gases. The present invention is directed to addressing this need in conjunction with potential implementation strategies for modifying the operation of the internal combustion engine.

In accordance with example embodiments of the present invention, a process for delivering air with elevated oxygen levels to the cabin of a vehicle is provided. The process includes intaking ambient air and separating the ambient air into a stream of oxygen-enriched air and a stream of oxygen-depleted air. The process also includes creating a modified oxygen-enriched air stream from the stream of oxygen-enriched air and ambient air from a second air intake in order to provide a specified concentration of oxygen to either the vehicle cabin or engine greater than that of ambient air.

In accordance with aspects of the present invention, the concentration of oxygen in the modified oxygen-enriched air stream provided to the cabin is in the range of about 21% to about 23.5%, where ambient air is approximately 20.95%. The modified oxygen-enriched air stream can include mixing the modified oxygen-enriched air stream with ambient air to a controlled ratio, and thereafter directing the controlled ratio into the cabin. The amount of oxygen present in the oxygen-enriched stream is monitored with a sensor with feedback capabilities, so as to enable safe operation should oxygen levels exceed a desired amount. For example, an acceptable range of oxygen presence is between about 26% and 40% oxygen by volume of air.

### BRIEF DESCRIPTION OF THE FIGURES

These and other characteristics of the present invention will be more fully understood by reference to the following detailed description in conjunction with the attached drawings, in which:

FIG. 1 is a diagrammatic illustration showing operation of the invention supplying oxygen-enriched air to a cabin;

FIG. 2 is a diagrammatic illustration showing operation of the invention supplying oxygen-enriched air to a cabin and an engine; and

FIG. 3 is a flowchart illustrating a process of creating and distributing oxygen-enriched air to a cabin and an engine of a vehicle.

### DETAILED DESCRIPTION

An illustrative embodiment of the present invention relates to a system and method that provides oxygen-enriched air to both the cabin and the internal combustion engine in an automobile. In accordance with the present invention, a desired increase in oxygen concentration is provided using oxygen-enriched air or a mixture of oxygen-enriched air and ambient air, thus resulting in a mixture including a greater percentage of oxygen than is present in ambient air. In accordance with an example embodiment of the present invention, oxygen-enriched air is produced using a gas separation membrane system or other devices utilizing pressure swing adsorption, vacuum swing adsorption, or combination thereof. Presently available single stage systems (e.g., such as those produced by Air Products, Generon, Monsanto, and others) provide a gas stream in which the oxygen concentration is about 35% (i.e., about 166% that of air) in the enriched stream, and greater concentrations can be obtained using multiple stage systems. Pressure Swing Adsorption Technology (PSA Technology) or Vacuum Swing Adsorption Technology (VSA Technology) can be used to generate oxygen from the air. With PSA Technology, at high pressures, a sieve adsorbs nitrogen, at low pressures sieves release nitrogen. As high pressure (about 70 psi) air is introduced, nitrogen is adsorbed and the remaining oxygen can be redirected to a storage tank or buffer. VSA Technology operates in a manner similar to PSA Technology but at near ambient temperatures and pressures wherein air is vacuumed through the separation process. Those of skill in the art will appreciate and understand the PSA and VSA Technology approaches for oxygen generation, such that additional details are not required. Several suitable membrane systems are discussed in U.S. Pat. No. 5,051,113, incorporated herein by reference. As would be appreciated by one skilled in the art, improved gas separation membranes are frequently introduced, and may be used in this invention. Regardless of the membrane system utilized with the present invention, the oxygen-enriched output is mixed with ambient air in the ratio required to produce an oxygen-enriched air stream which will provide the desired oxygen concentration in the cylinder. The ratio at which ambient air is mixed with the oxygen-enriched air from the membrane system can be varied over a wide range to permit the percentage of oxygen, in the gas that is input to the cylinder to be anywhere between the oxygen concentration in ambient air and that in the oxygen-enriched air stream. The oxygen level in the mixed stream can be regulated using a valve system to control the flowrate of the oxygen-enriched and standard air streams. Furthermore, an oxygen sensor can be installed in mixed air stream to monitor the oxygen level, and subsequently provide feedback to the valve control system for aid in providing a desired volume fraction of oxygen.

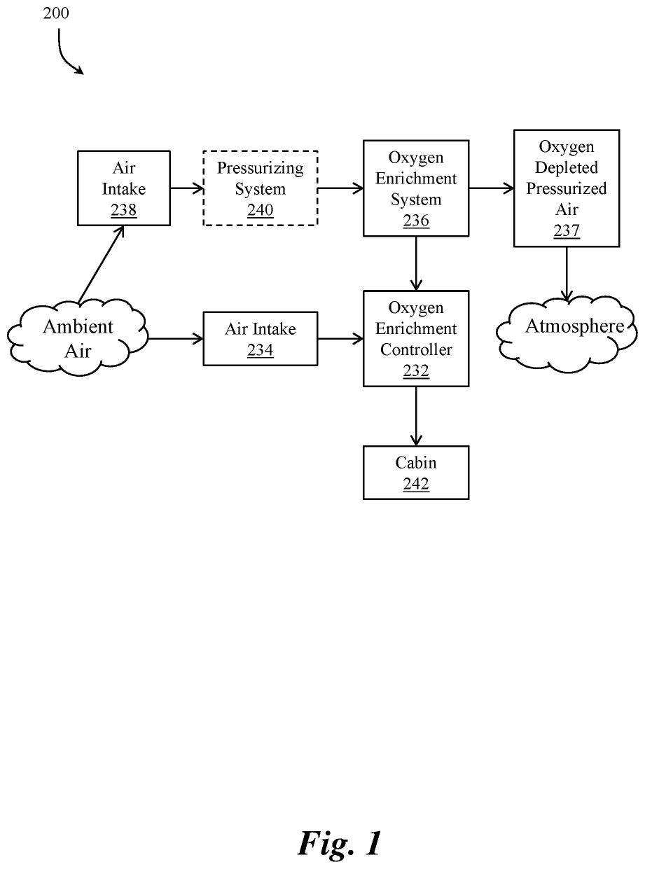

FIGS. 1 through 3, wherein like parts are designated by like reference numerals throughout, illustrate an example embodiment or embodiments of improved operation for combustion engines, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed in a manner still in keeping with the spirit and scope of the present invention.

FIG. 1 depicts a diagrammatic illustration of a vehicle oxygen-enriched cabin air system 200 for controlling the concentration of oxygen introduced to the vehicle cabin. As would be appreciated by one skilled in the art, systems for monitoring and/or controlling the injection of oxygen and fuel are readily available or constructed; and in many circumstances are found in conventional mechanical or pneumatic (e.g., air powered) injection systems.

In accordance with an example embodiment of the present invention, a gas stream input to intake manifold is oxygen-enriched, and the extent of oxygen-enrichment is controlled by an oxygen concentration controller 232. As depicted in FIG. 1, ambient air is received by the air intake 234 and input into the oxygen concentration controller 232. The oxygen concentration controller 232 also receives oxygen-enriched air as a second input from the oxygen-enrichment system 236. In the illustrated embodiment of FIG. 1, oxygen-enrichment system 236 acts as a membrane air separator (e.g., such as that shown in U.S. Pat. No. 5,051,113) that includes a semi-permeable membrane having a higher permeability for oxygen than for nitrogen. In operation, the membrane of the oxygen-enrichment system 236 separates input ambient air (from air intake 238 and/or air pressurizing system 240) into an oxygen-enriched air stream, which is input to the oxygen concentration controller 232 and an oxygen-depleted air stream 237, which is exhausted to the atmosphere. Alternatively, the air stream may be exhausted through an energy recovery turbine so that the energy contained in the oxygen-depleted air stream 237 can be used to pressurize ambient air in the air pressurizing system 240.

In accordance with an example embodiment of the present invention, the air intakes 238, 234 are separate systems. As separate systems, the air intake 238 provides a standalone supply of air to the oxygen-enrichment system 236, and the air intake 234 provides the air supply to the engine 244 (via the oxygen concentration controller 232). Thereafter, the oxygen concentration controller 232 supplies high pressure fuel (most likely liquid, but can be gaseous) to the combustion chamber (via the air intake manifold) and in certain embodiments the air pressurizing system 240 supplies high pressure ambient air to the oxygen-enrichment system 236. As would be appreciated by one skilled in the art, the air pressurizing system 240 could be omitted without departing from the scope of the present invention. Additionally, the air intakes 238, 234 can be a single unit with multiple outputs. For example, the air intakes 238, 234 can start as one intake that splits to provide separate air streams to the air pressurizing system 240 and the oxygen concentration controller 232.

In typical operating ranges the oxygen-enriched air stream from oxygen-enrichment system 236 has an oxygen concentration of about 35% (although improved air separation membranes may be produced which would allow even higher concentrations from a single stage system), and thus contains a considerably lower percentage of nitrogen than does ambient air. The oxygen concentration controller 232 mixes the gas streams from the two inputs (e.g., the ambient air from air intake 234 and the oxygen-enriched air from the oxygen-enrichment system 236) and provides the oxygen-enriched air output to the cabin 242. Ideally, the oxygen-enriched air output to the cabin 242 will have a desirable oxygen concentration in the range of about 21% to 23.5% oxygen and, preferably 22% to 35% oxygen.

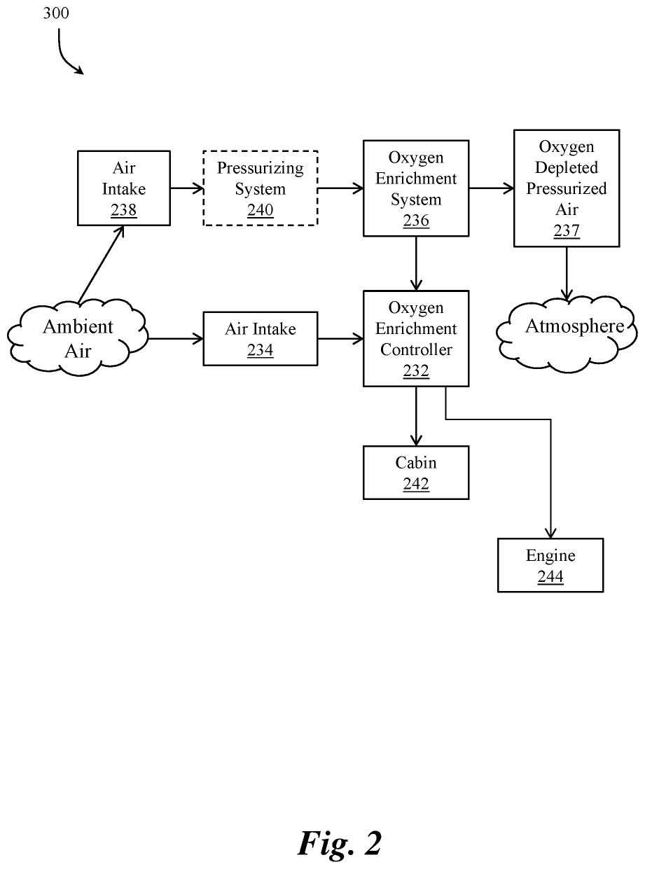

FIG. 2 depicts a vehicle oxygen-enriched cabin air system 300 similar to the combustion system 200 depicted in FIG. 1 with a desired portion of the oxygen enhanced air provided to both the cabin 242 and an engine 244 motivating the vehicle in which the cabin 242 is situated. The vehicle oxygen-enriched cabin air system 300 utilizes the oxygen concentration controller 232 to manage inflow of oxygen-enriched air from the oxygen-enrichment system 236 and ambient air from the air intake 234, and controls supply and distribution of the oxygen-enriched air to the cabin 242 and the engine 244 of the vehicle.

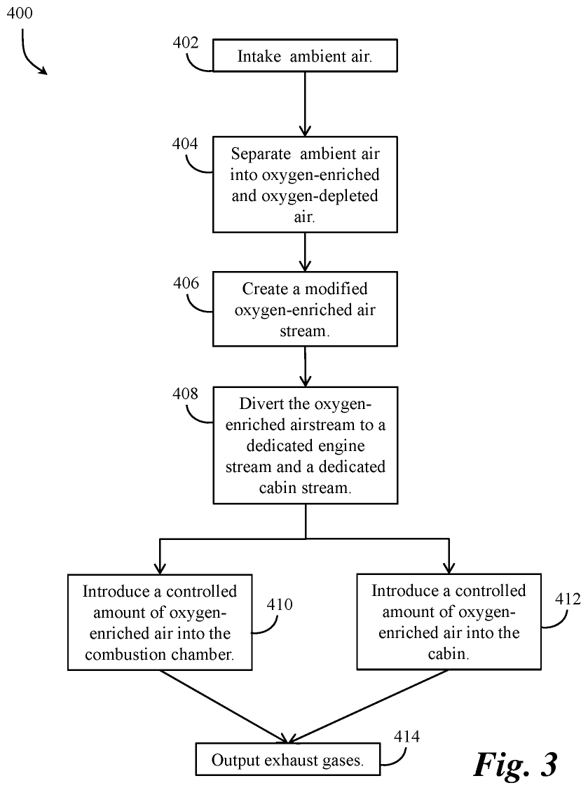

In accordance with aspects of the present invention, as depicted in the flowchart of FIG. 3, the oxygen-enriched cabin air system 300 implements a process 400 for providing elevated oxygen concentrations to the vehicle in which the system resides. The system 300 intakes and pressurizes ambient air from external to the vehicle through a first air intake (step 402). The pressurized ambient air is separated into a stream of oxygen-enriched air and a stream of oxygen-depleted air (step 404). At a source, a modified oxygen-enriched air stream is created from the stream of oxygen-enriched air and ambient air from a second air intake (step 406). The modified oxygen-enriched air stream is diverted from the source into a dedicated engine modified oxygen-enriched air stream and a dedicated cabin modified oxygen-enriched air stream (step 408). A controlled amount of the dedicated engine modified oxygen-enriched air stream is introduced into the combustion chamber and increasing an oxygen concentration in the combustion chamber during the combustion cycle to a concentration greater than that of ambient air external to the vehicle (step 410). A controlled amount of the dedicated cabin modified oxygen-enriched air stream is introduced into the vehicle cabin and increasing an oxygen concentration in the vehicle cabin to a desired concentration threshold greater than that of ambient air external to the vehicle (step 412). Exhaust gases are output from the system 300 (step 414).

The concentration of oxygen in the dedicated cabin modified oxygen-enriched air stream and the dedicated engine modified oxygen-enriched air stream can be in the range of about 21% to about 23.5%. The concentration of oxygen in the dedicated cabin modified oxygen-enriched air stream and the dedicated engine modified oxygen-enriched air stream can be in the range of about 21% to about 40% oxygen in air. The oxygen concentration is monitored by a sensor that provides feedback to the controller to maintain a desired oxygen concentration.

The step of introducing the controlled amount of the dedicated cabin modified oxygen-enriched air stream into the cabin (step 412) or the dedicated engine modified oxygen-enriched air stream into the combustion chamber (step 410) comprises mixing the modified oxygen-enriched air stream with ambient air in a controlled ratio, and thereafter directing the controlled ratio into the combustion chamber during the intake cycle and before the combustion cycle. The source is provided by the implementation of two parallel oxygen-enrichment systems.

The system 300, 400 can further include a parallel oxygen controller configured to receive the oxygen-enriched air stream input from the first oxygen-enrichment system, receive the second oxygen-enriched air stream from the second oxygen-enrichment system, meter a desired amount of oxygen into the air intake manifold from the oxygen-enriched air stream input, and instruct the second oxygen-enrichment system to replenish the second oxygen-enriched air stream while the enriched air stream input from the first oxygen-enrichment system is being used to supply airflow to the air intake manifold. When a store of oxygen in the first oxygen-enrichment system is depleted, the parallel oxygen controller switches over a supply to the second oxygen-enriched air stream such that the second oxygen-enrichment system provides oxygen-enriched air to the air intake manifold.

The system 300, 400 can further include a parallel oxygen controller configured to receive oxygen-enriched air provided by a tank of pressurized oxygen, or from a process of electrolysis of water, or from pressure or vacuum swing adsorption. A safety shut-off mechanism that shuts off oxygen flow when oxygen concentrations exceed predetermined levels can be provided. The predetermined levels would be determined by those of skill in the art not to exceed conditions that are dangerous for occupants of the vehicle. For example, it is understood that exceeding 40% oxygen by volume increases the risk of fire ignition and also oxygen toxicity, and therefore oxygen concentrations can be set not to exceed a predetermined level of 40% oxygen by volume in the cabin. However, oxygen therapy for patients recovering from surgery use up to a 35% oxygen concentration.

The oxygen-enriched air can be configured to be introduced into a stock ventilation system of the vehicle, or as a modular after-market add-on system. The oxygen-enriched air can be introduced to the cabin through a dedicated port. A sensor can be installed in the cabin to monitor the oxygen levels, wherein the sensor records oxygen levels over time.

A vehicle operator control can be provided wherein the vehicle operator is enabled to specify whether available excess oxygen is to be preferentially routed to the cabin or to the engine. Oxygen-enriched air above the desired concentration threshold can be directly introduced to the cabin in order to mix with the standard air already contained therein, thus resulting in a rapid increase to oxygen level in the cabin. A mathematical algorithm can be used to predict the amount of hyper-concentrated air that can be mixed with standard air contained in the cabin to achieve the desired concentration threshold amount of oxygen in the cabin. Variations on such algorithms would be appreciated by those of skill in the art, and therefore not required in detail herein. A mathematical algorithm can also be used to predict optimal oxygen usage between the cabin and engine in order to maximize the benefits of each.

The desired oxygen concentration threshold in the cabin can be modulated based on operation times, length of operation, and driver behavior. Air removed from the cabin can be routed through the oxygen-enriching device when air present in the output of the vehicle's ventilation system is at an already elevated level. A sensor can be used to determine the amount of oxygen in the air being discharged from the vehicle's cabin.

A proportion of mixing between the oxygen-elevated air stream and the ambient air stream is controlled by a valve system, by modulating the pressure of the individual supply lines, or by modulating a temperature of the individual supply lines. A mixing between the oxygen-enriched air stream and the ambient air stream is enhanced by, including a mixing chamber with sufficient volume, pulsing (through pressure variations) the supply lines, and/or introducing swirl or irregular motion in the supply lines when mixed so as to not introduce pockets of extreme oxygen concentration.

As would be appreciated by one skilled in the art, other embodiments will be within the scope of the present invention. As would be appreciated by one skilled in the art, the various aspects depicted and discussed with respect to FIGS. 1-2 can be combined into a single system. Combining all the functional elements form all three figures would provide the greater operational flexibility and the greatest chance at improvement, however, some of the implementations would be expensive with only minor benefits, such that some elements may be excluded.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A process for providing elevated oxygen concentrations to a vehicle, the process comprising:
intaking ambient air from external to the vehicle through a first air intake, establishing an ambient air stream;
separating the ambient air stream into a stream of oxygen-enriched air and a stream of oxygen-depleted air;
at a source, creating a modified oxygen-enriched air stream from the stream of oxygen-enriched air and ambient air from a second air intake;
an oxygen concentration controller managing to divert the modified oxygen-enriched air stream from the source into a dedicated engine modified oxygen-enriched air stream and a dedicated cabin modified oxygen-enriched air stream;
introducing a controlled amount of the dedicated engine modified oxygen-enriched air stream into a combustion chamber of an engine of the vehicle and increasing an oxygen concentration in the combustion chamber during a combustion cycle to a concentration greater than that of ambient air external to the vehicle;
introducing a controlled amount of the dedicated cabin modified oxygen-enriched air stream into a vehicle cabin and increasing an oxygen concentration in the vehicle cabin to a desired concentration threshold greater than that of ambient air external to the vehicle; and
outputting exhaust gases.

2. The process of claim 1, wherein a concentration of oxygen in the dedicated cabin modified oxygen-enriched air stream and the dedicated engine modified oxygen-enriched air stream is in a range of about 21% to about 23.5%.

3. The process of claim 1, wherein a concentration of oxygen in the dedicated cabin modified oxygen-enriched air stream and the dedicated engine modified oxygen-enriched air stream is in a range of about 21% to about 40% oxygen in air.

4. The process of claim 1, wherein the oxygen concentration is monitored by a sensor that provides feedback to the oxygen concentration controller to maintain a desired oxygen concentration.

5. The process of claim 1, wherein the introducing the controlled amount of the dedicated cabin modified oxygen-enriched air stream into the vehicle cabin or the dedicated engine modified oxygen-enriched air stream into the combustion chamber comprises mixing the modified oxygen-enriched air stream with ambient air in a controlled ratio, and thereafter directing the controlled ratio into the combustion chamber during the intake cycle and before the combustion cycle.

6. The process of claim 1, wherein the source is provided by implementation of two parallel oxygen-enrichment systems.

7. The process of claim 6, further comprising a parallel oxygen controller configured to:
receive a first oxygen-enriched air stream input from a first oxygen-enrichment system;
receive a second oxygen-enriched air stream from a second oxygen-enrichment system;
meter a desired amount of oxygen into an air intake manifold from the first oxygen-enriched air stream input; and
instruct the second oxygen-enrichment system to replenish the second oxygen-enriched air stream while the enriched air stream input from the first oxygen-enrichment system is being used to supply airflow to the air intake manifold.

8. The process of claim 7, wherein when a store of oxygen in the first oxygen-enrichment system is depleted, the parallel oxygen controller then switches over a supply to the second oxygen-enriched air stream such that the second oxygen-enrichment system provides oxygen-enriched air to the air intake manifold.

9. The process of claim 1, wherein oxygen-enriched air is provided by a tank of pressurized oxygen.

10. The process of claim 1, wherein oxygen is provided from electrolysis of water.

11. The process of claim 1, wherein the oxygen is provided by a device utilizing pressure swing adsorption.

12. The process of claim 1, wherein the oxygen is provided by a device utilizing vacuum swing adsorption.

13. The process of claim 1, further comprising a safety shut-off mechanism that shuts off oxygen flow when oxygen concentrations exceed predetermined levels.

14. The process of claim 1, wherein oxygen-enriched air is introduced into a stock ventilation system of the vehicle.

15. The process of claim 1, wherein oxygen-enriched air is introduced to the vehicle cabin through a dedicated port.

16. The process of claim 1, wherein a sensor is installed in the vehicle cabin to monitor oxygen levels, and wherein the sensor records oxygen levels over time.

17. The process of claim 1, further comprising a vehicle operator control, and wherein a vehicle operator is enabled to specify whether available excess oxygen is to be preferentially routed to the vehicle cabin or to the engine.

18. The process of claim 1, wherein oxygen-enriched air above the desired concentration threshold is directly introduced to the vehicle cabin in order to mix with the standard air already contained therein, thus resulting in a rapid increase to oxygen level in the vehicle cabin.

19. The process of claim 1, wherein a mathematical algorithm is used to predict an amount of hyper-concentrated air that can be mixed with standard air contained in the vehicle cabin to achieve the desired concentration threshold amount of oxygen in the vehicle cabin.

20. The process of claim 1, wherein a mathematical algorithm is used to predict optimal oxygen usage between the vehicle cabin and engine in order to maximize benefits of each.

21. The process of claim 1, wherein the desired oxygen concentration threshold in the vehicle cabin is modulated based on operation times, length of operation, and driver behavior.

22. The process of claim 1, wherein air removed from the vehicle cabin is routed through an oxygen-enrichment system when air present in the output of a ventilation system of the vehicle is at an already elevated level.

23. The process of claim 1, wherein a sensor is used to determine an amount of oxygen in the air being discharged from the vehicle cabin.

24. The process of claim 1, wherein a proportion of mixing between the oxygen-elevated air stream and the ambient air stream is controlled by a valve system, by modulating the pressure of the individual supply lines, or by modulating a temperature of individual supply lines.

25. The process of claim 1, wherein a mixing between the oxygen-enriched air stream and the ambient air stream is enhanced by, including a mixing chamber with sufficient volume, pulsing, through pressure variations, the supply lines, and/or introducing swirl or irregular motion in the supply lines when mixed so as to not introduce pockets of extreme oxygen concentration.
